(19) United States
(12) Patent Application Publication
(10) Pub. No.: US 2004/0234940 A1
(43) Pub. Date: Nov. 25, 2004

(54) System

(76) Inventors: Hein Van Der Steen, Franklin, KY (US); Lalantha R. Abeydeera, Franklin, KY (US); Jon E. Anderson, Franklin, KY (US)

Correspondence Address: HUNTON & WILLIAMS LLP, INTELLECTUAL PROPERTY DEPARTMENT, 1900 K STREET, N.W., SUITE 1200, WASHINGTON, DC, 20006-1109, US

(21) Appl. No.: 10/481,249

(22) PCT Filed: Jun. 18, 2002
(86) PCT No.: PCT/GB02/02792

(30) Foreign Application Priority Data
Jun. 18, 2001 (GB) 0114849.3

(51) Int. Cl. A01N 1/02; C12M 1/34

(52) U.S. Cl. 435/2; 435/287.1

(57) ABSTRACT

A micro chamber arrangement for development of oocytes, fertilisation and embryo culture. Objects are kept in a micro environment. The system monitors and regulates the environment for the objects and incorporates regular quality control. The device is very suitable for large scale embryo production. The arrangement can be used for follicle sorting, follicular growth, oocyte maturation, oocyte enucleation, in vitro fertilisation, cloning, nuclear transfer, genetic modification, embryo culture, embryo encapsulation and embryo transport.

The present invention relates to a system for in vitro cell culture, in particular embryo production (IVP) from harvesting and growth of ovarian follicles, maturation and fertilisation of oocytes up to embryo culture and transport in aquatic species and mammals, including human. In particular it relates to a highly automated system utilising micro-chamber technology (MCT), robotics, video image analysis, biochemical analysis and software.

A commercial embryo production system consists of all or some of the following steps

1) The isolation of primordial follicles, preantral follicles or cumulus-oocyte complexes (COCs) present in ovaries. A COC is an intact immature (germinal vesicle stage) oocyte completely surrounded by cumulus cells, extracted from antral follicles.

2) Growth of primordial or preantral follicles to antral follicle stage

3) In vitro maturation (IVM) of oocytes or COCs

4) Production of one cell embryos from mature oocytes by one of the following methods

a) In vitro fertilisation (IVF) or

b) Nuclear Transfer (NT)

c) IVF followed by NT

5) In Vitro embryo culture (IVC)

6) Embryo transport from the laboratory to the location of the recipient females

7) (Non) Surgical Embryo Transfer ((N)SET) to recipient female

All of the individual steps above have been performed in several species, and are potentially relevant for mammals. However in fish, although the first five steps could be relevant, it is most likely that the process would start with the collection of oocytes (eggs) and sperm cells from mature individuals. The main objective in aquatic species (fish, crustaceans, and molluscs) would be IVF and culture of the embryos through the first developmental stages. Additional techniques applicable to this microchamber technology are centrifugation or treatment of the object. Examples of such treatments are

changing the chemical composition (adding hormones, enzymes etc.)

changing the temperature

electrical currents.

For instance in fish a temperature shock can be applied at a stage where the oocyte is still 2n. This blocks the meiotic progression of a haploid (n) oocyte and the resulting fertilised egg (Zygote) is 3n. The temperature shock can also be applied between the one and two cell stage to produce tetraploids. Timing and duration of this treatment in relation to the point of fertilisation and the cell cycle is vital. Micro-chamber technology will be the tool to do this.

Progress in embryo research is slow as the process is labour intensive and the validation phase (producing live born individuals) is expensive and time consuming. The main problem of current embryo production systems is the low efficiency. The development of systems and methods to address these problems would assist in the production of clones (no genetic variation) and larger full sib families (50% genetic variation.) It could also increase the rate of genetic improvement in livestock breeding programs focusing on efficiency of production and product quality. The most suitable genotypes could be produced independent of the genetic potential of the recipient female, e.g. embryos produced from lines developed for performance of the terminal generation, e.g. in pigs sire line embryos transferred to recipient dam line females or beef embryos transferred to dairy cow recipients. Specific genotypes could also be conserved and preserved.

Another benefit of the proposed microchamber technology is that the number of progeny per animal (male and female) especially in valuable/genetically superior females with low natural reproductive rates could also be increased and thus make better use of these valuable individuals. In addition, using sperm separated according to sex chromosome (X or Y) could produce single sex progeny. Such methods applied to microchamber technology would also eliminate the need for large scale multiplication in order to supply a large commercial base from a relatively small genetic nucleus where the genetic improvement programs are implemented

Successful embryo production procedures require knowledge of the environment that oocytes, spermatozoa, cells and embryos need for optimal development. Methods such as the described microchamber technology need to be developed to provide this environment in an efficient way without imposing stress on said oocytes, spermatozoa, cells and embryos. For human applications and for the production of founder animals through genetic modification (GM), the efficiency of embryo technology techniques is less relevant. The multiplication of GM-founders and most agricultural applications require high levels of efficiency.

Existing embryo technology is based on fine-bore glass pipettes and standard petri dishes and is labour intensive due to manual individual handling and magnified observation. As a result progress is slow. Culture systems require relatively large quantities of medium and supplements, which increases costs for the production of embryos from relatively few oocytes per ovary currently obtained using traditional systems. Another new technology recently reported is the development of embryo fluidic channel systems by the group of Beebe and Wheeler from the University of Illinois. (Raty S, et al. 2001; Walters et al 2001.) However, this technology will be difficult to scale up. It is also less suitable for the frequent monitoring of quality required in embryo production and for the incorporation of robotics and DISK technology that allows centrifugation, facilitates automation and localisation of individual objects.

An improved IVP system needs to be large scale, relatively inexpensive, robust enough to be easy to implement in a commercial laboratory, and be able to create the required microenvironment for the developing embryos. It must also be able to monitor the quality of development and to identify oocytes and embryos with the suitable developmental capacity to enable separation on this basis. In addition it must be flexible with respect to the incorporation of other technology. We describe herein novel methods and systems for embryo production which address the above described problems.

Thus, the present invention provides apparatus for handling and/or treatment of follicles, oocytes and/or embryos comprising at least one of:

(a) a chamber containing a plurality of sieve elements arranged in succession within the chamber, wherein each successive sieve element has pores of a smaller dimension than those in the preceding sieve element, connected to a pump to maintain a circulatory flow of medium, wherein said sieve elements separate primordial follicles, Preantral Follicles or Cumulus-Oocyte-Complexes (COCs) from ovarian debris and sort the follicles according to size; and

(b) a micro chamber arrangement containing a plurality of microchambers, each optionally comprising one or more sieve elements

The microchamber can either have no base, or a base formed by a sieve element. If the microchambers lack a base then the arrangement is placed so there is a very narrow space between the bottom of the container holding the medium and the chamber walls to ensure that the follicles, oocytes or embryos can not escape.

In one preferred embodiment the micro chamber arrangement incorporates a pump that controls recirculation of culture medium. Thus, this makes it possible to gradually change the composition of the medium, to filter out toxic components and to control the level of favourable factors produced by the developing objects. This effectively determines the degree of follicle, oocyte or embryo co-culture.

In another preferred embodiment the micro chambers each comprise a sieve element having adjustable pore dimensions, this allows, e.g. sorting based on quality assessment and allows for the removal of low quality follicles, oocytes or embryos or the transfer of good quality follicles, oocytes or embryos as needed.

In a further preferred embodiment the micro chamber arrangement incorporates a second pump system for the introduction of spermatozoa into the micro chambers as desired. The walls of each micro chamber may also contain holes to allow the circulation of medium between chambers.

In yet another preferred embodiment the micro chamber arrangement comprises means for encapsulation of chosen embryos within a protective coating.

In another preferred embodiment the walls between the microchambers contain holes so that medium is permitted to flow in and out of each individual microchamber from the sides and/or the top and bottom.

The arrangements as described herein can be used in the following methods:

1. Sieve system

enzymatic treatment of ovarian tissue

ovarian tissue culture

batch culture of follicles

sorting of follicles from debris

2. Micro-chambers

(a) follicular growth and development

(b) Removal of cumulus cells from antral stage COCs

(c) Development of oocytes (IVM)

(d) In vitro fertilisation (IVF)

(e) Nuclear transfer (NT)

(f) Embryo culture (IVC)

(g) Splitting embryos with the addition of enzymes to the media

(h) Sorting of objects according to size

(i) Selection of objects according to quality

(j) Normal cell culture

The micro chamber arrangement can be in the format of a DISK or plate, which facilitates identification of the individual chambers, and allows for centrifugation and individual quality control based on image analysis. Quality assessment can be carried out by the incorporation of a video camera connected to an image capturing device such as a microscope. The centrifugation allows precise positioning of the object and enables micro-injection for cloning or genetic modification to be carried out.

Another application of micro-chamber technology is in the embryo freezing process that involves a centrifugation step. Since pig embryos contain large amounts of lipid in their cytoplasm that prevents successful cryopreservation, centrifugation can be used to remove the lipids from the individual cells of the embryo (Beebe LFS, et al. 2000). The use of micro-chamber technology in a DISK format will enable embryos to be centrifuged at the end of culture, which is immediately prior to the cryopreservation step.

The disk can have a circular or rectangular shape. The rectangular shape may be better for robotic handling. The disk can spin around the center point, with the centrifugal force depending on the distance of the individual micro chamber from the center. An alternative is to put the disk against the side wall of a centrifuge, i.e. parallel with the axis of rotation, in which case each micro-chamber will experience the same centrifugal force.

The combination of follicle, oocyte and/or embryo development in micro chambers and a DISK format allows for the handling of about 10,000 (or more or less) oocytes/embryos per disk which leads to large scale and low cost production of embryos in mammals and larvae in aquatic species. The incorporation of DISK technology and robotics will lead to robust systems for commercial use. Disks with oocytes/embryos in micro chambers can be monitored while undergoing IVM, IVF, and/or IVC to allow for individual quality of the oocytes/embryos to be assessed based on image analysis, near infra-red (NIB) and/or other techniques. Features such as size, shape, structure, density, metabolic activity and developmental changes over time can be used as quality parameters. This process can also be used for culturing other cells and cells lines. Oocytes/embryos that meet quality targets can continue the developmental process until the desired stage is reached, at which time transfer to recipient females (mammals) or further culture (aquatic species) can occur.

For the application of this technology to IVF, one would determine the optimal stage of in-vitro maturation at which the sperm cells are added to the micro-chambers. After x minutes of co-culture one can then flush out the non-attached/penetrated sperm cells. After y minutes the process can then be repeated. The second wave of semen will only have an impact if fertilisation did not take place initially. One can optimise the sperm concentration, the duration of co-culture (x), the interval between co-culture periods (y) and the number of co-culture periods in order to improve the results. Such improvements would include the reduction of poly-spermy and increases in sperm penetration and zygote cleavage rates.

Suitably, the apparatus of the present invention is controlled by a computer system. This allows for automation of the process and handling of the follicles, oocytes and/or embryos, as well as allowing for “feedback” control of the characteristics of the cultured follicles, oocytes or embryos and/or properties of the culture medium.

### BRIEF DESCRIPTION OF THE DRAWINGS

The FIGS. 1 to 15 describe the main features of the system. Modifications and additions can be made without changing the basic concept of the invention.

FIG. 1 shows a vertical cross section of apparatus that allows enzymatic treatment and the sorting of processed ovarian material into debris and different sizes of follicles and Cumulus-Oocyte-Complexes (COCs);

FIG. 2 is an overhead view of the sieves of the sorting arrangement (FIG. 1);

FIG. 3 is a section of a micro chamber arrangement illustrating chamber design and configuration;

FIG. 4 is an overhead view of elements forming the bottom layer of the individual micro chamber;

FIG. 5 shows a micro chamber arrangement in the format of a DISK;

FIG. 6 shows a vertical cross section of a small part of a micro chamber arrangement used for static growth/maturation/culture of the oocytes/embryos;

FIG. 7 shows a vertical cross section of a small part of a micro chamber growth/maturation/culture arrangement with recirculation of the medium;

FIG. 8 shows a vertical cross section of a micro chamber arrangement that leads to sorting based on size;

FIG. 9 illustrates the removal of cumulus cells from the COCs;

FIG. 10 shows a vertical cross section of arrangement for in vitro fertilisation;

FIG. 11 is an overhead view of a modified micro chamber that allows fixation of the object for microinjection or biopsy;

FIG. 12 shows a vertical cross section of a micro chamber arrangement used for “disaggregation” of blastomeres to produce identical embryos;

FIG. 13 is diagram that illustrates selection based on quality;

FIG. 14 illustrates the encapsulation of embryos;

FIG. 15 illustrates a cross-sectional view of a micro-chamber technology device with shared medium volume.

The present invention will now be described with reference to the accompanying drawings which describe a handling device for follicles, oocytes and embryos. The device can be used for in vitro cell culture, the isolation of follicles, in vitro growth of follicles, in vitro maturation of COCs, in vitro fertilisation, nuclear transfer, in vitro embryo culture, encapsulation and transport.

Isolation of Follicles

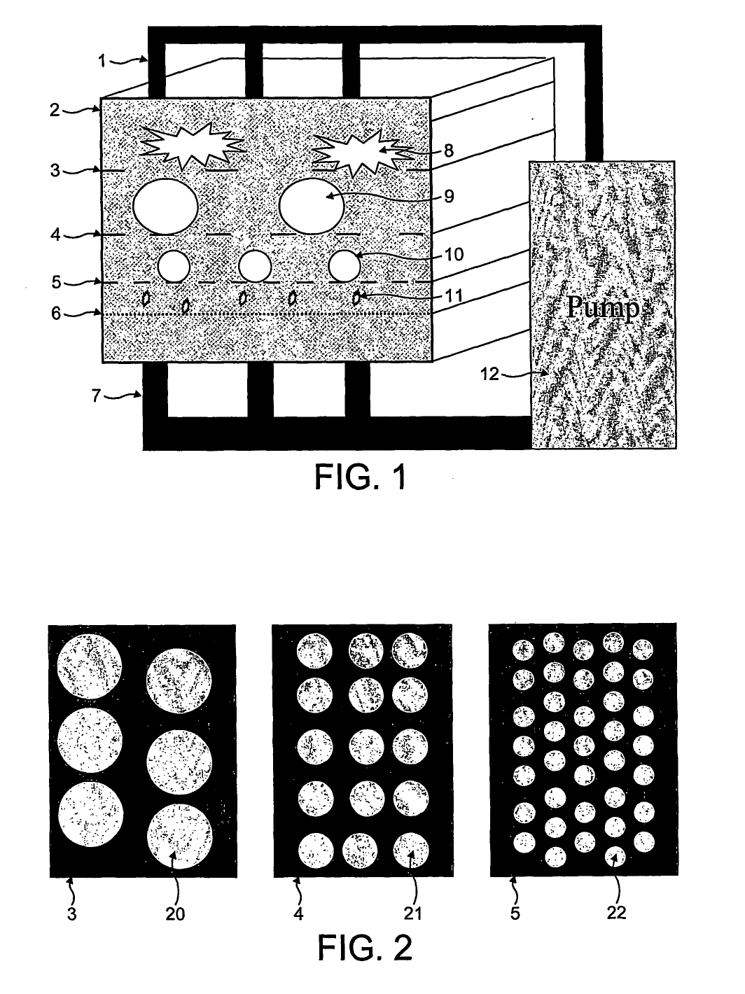

Enzymatic and/or mechanical procedures can be used to harvest primordial and preantral follicles from the ovaries of a female mammal. (Figueiredo et al., 2000; Hirao et al., 1994; Wu et al., 2001; McCaffery et al., 2000; Telfer et al., 2001.) The follicles and debris can be suspended in a medium and are then available for further processing by the follicle and ovary debris sorting device (FIG. 1). This leads to the production of many isolated follicles per donor female.

Depending on the age of the animal, ovarian cortex contains large numbers of primodial an/or preantral follicles. To recover these follicles from ovaries, enzymatic and/or mechanical procedures can be used. Mechanical isolation is carried out on thin ovarian slices or small pieces by using fine forceps and/or needles. This is a tedious and time consuming process. Enzymatic isolation involves both mechanical chopping or mincing of ovarian tissue followed by enzymatic (collagen) digestion for a specific period of time. After digestion, contents can pass through a series of filters with various sieve sizes to remove debris and retain the desired follicles.

A chamber (2) with sieve elements (3, 4, 5, for detail see FIG. 2) can be used for enzymatic digestion and to separate follicles (9,10) from ovarian debris (8, 11). The chamber (2) can be moved by mechanical methods in horizontal and/or vertical directions while containing some culture medium. This will allow sorting based on size and removal of ovarian debris. Gentle movements, horizontal and/or vertical, will help to maximise the filtration process. If a larger follicle rests on a sieve, it can prevent any smaller objects (small follicles and debris) from passing through. A slight agitation would resuspend the contents and allow efficient separation. The ovarian material is placed in the top layer of the chamber. The medium flows through the chamber (2) from the multiple inlet (1) to the multiple outlet (7). The top sieve element (3) will retain the debris (8) that is larger than individual follicles (9,10). This large material can return to the ovary treatment stage to yield additional follicles. The lower layers of the separation device will hold follicles (9, 10) of different sizes and the small debris (11) will end up at the bottom. The number of sieve elements can be altered to split the follicles (9,10) into one or more size groups. The medium may contain enzymes to further break up the ovarian tissue into the individual follicles. Once sorted by size, the separated follicles can go to different devices for further processing. The flow rate, controlled by a pump (12), determines the amount of force applied to the sorting device for the separation of debris from the follicles. The pump (12) also contains a filter that collects the debris. A filter (6) can also be built in at the bottom of the chamber (2). The diameter of the medium outlet (7) is larger than the diameter of the openings of the last sieve (5) or filter (6). The inlets (1) and outlets (7) are evenly spread over the top and bottom of the device to give a uniform flow rate across the total chamber (2). The example shown consists of 6 separate parts that snap together into one unit, i.e. the top with the inlets (1), three sieves (3,4,5), one filter (6) and the bottom with the medium outlets (7). The sieve elements (3,4,5) have openings (20,21,22) of decreasing dimension.

Micro-Chambers

Follicles that have been separated by the sorting device (FIG. 1) are suspended in a medium of a certain viscosity. The combined volume of follicles and medium is approximately equal to the volume of the micro chambers of one arrangement. This medium containing the follicles is spread evenly over the surface of the arrangement maximising the chance that one chamber will contain only one follicle. The follicles may enter the microchambers through capillary force, suction, medium flow, or any other mechanism. One 10 cm by 10 cm arrangement could contain 10,000 micro chambers. In that case the medium would contain less than 10,000 follicles. The ratio between number of follicles and number of chambers, the viscosity of the medium and the method of spreading the medium with follicles over the arrangement needs to be optimised. Follicle characteristics such as size, shape, outer membrane structure and, density, along with size of the oocyte can be used to identify and sort follicles into different classes for further growth and maturation.

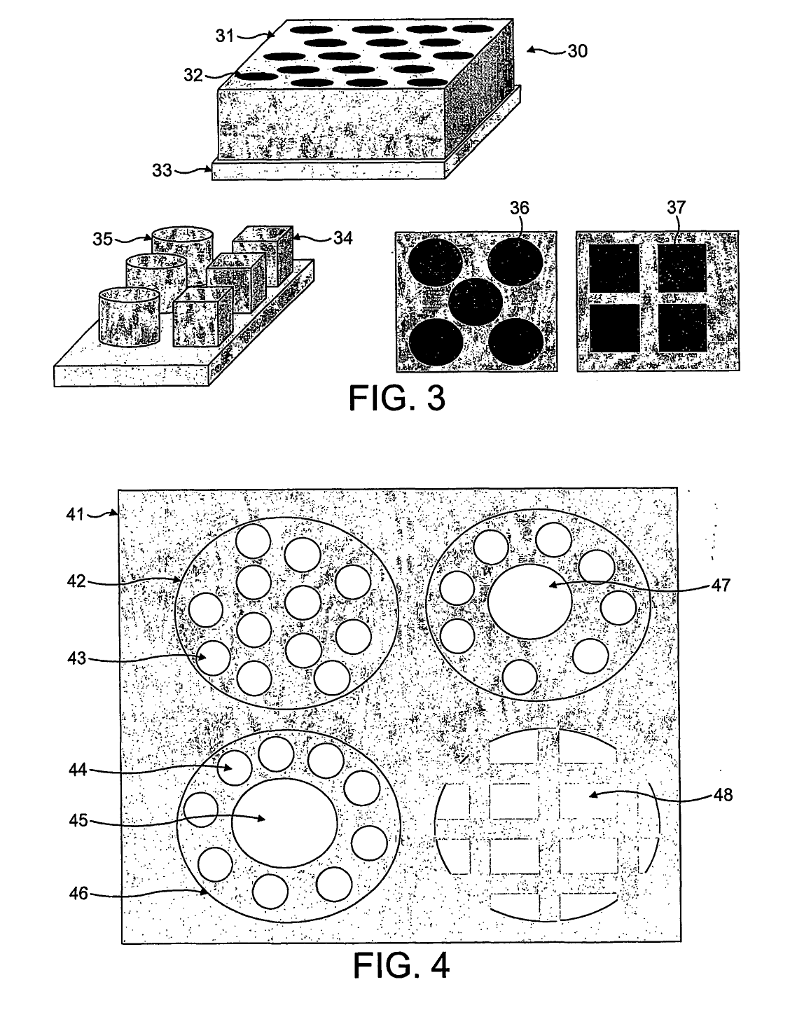

The isolated follicles will go to a micro-chamber arrangement (FIG. 3) for further development. The arrangement (30) can consist of either one part (31) or two parts (31) and (33). (31) consists of a series of microchambers, while (33) consists of a porous material or a series of sieve elements arranged below the microchambers. The height of part (31) can be between 200 and 5000 micron, probably close to 500 micron. The dimensions of the surface could be anything between 1 cm by 1 cm and 25 cm by 25 cm or whatever size is needed given the required capacity. (31) can be circular (35) or square (34). FIG. 3 also provides overhead views of square (37) or circular (36) micro chamber arrangements. The dimensions going from top to bottom may vary, i.e. wider at the top than at the bottom.

If the arrangement consists of only one part (31), then the microchamber is placed so that there is only a narrow gap between the bottom of the walls of the chamber and the base of the container holding the culture medium.

If the arrangement consists of two parts, (31) sits on top of (33) which forms the bottom of the chambers (32). The bottom (41) of each individual chamber (42,46), as visualised in FIG. 4, contains square (48) or circular (43, 44) holes of equal size (or any other porous arrangement with similar effect) or with one additional larger hole (45, 47). Arrangements with chambers that only contain small holes are referred to as type I arrangements while those that also contain a larger hole are referred to as type II development of the oocyte/embryo. Materials such as plastics stainless steel, silicon and other materials used in tissue culture can be considered.

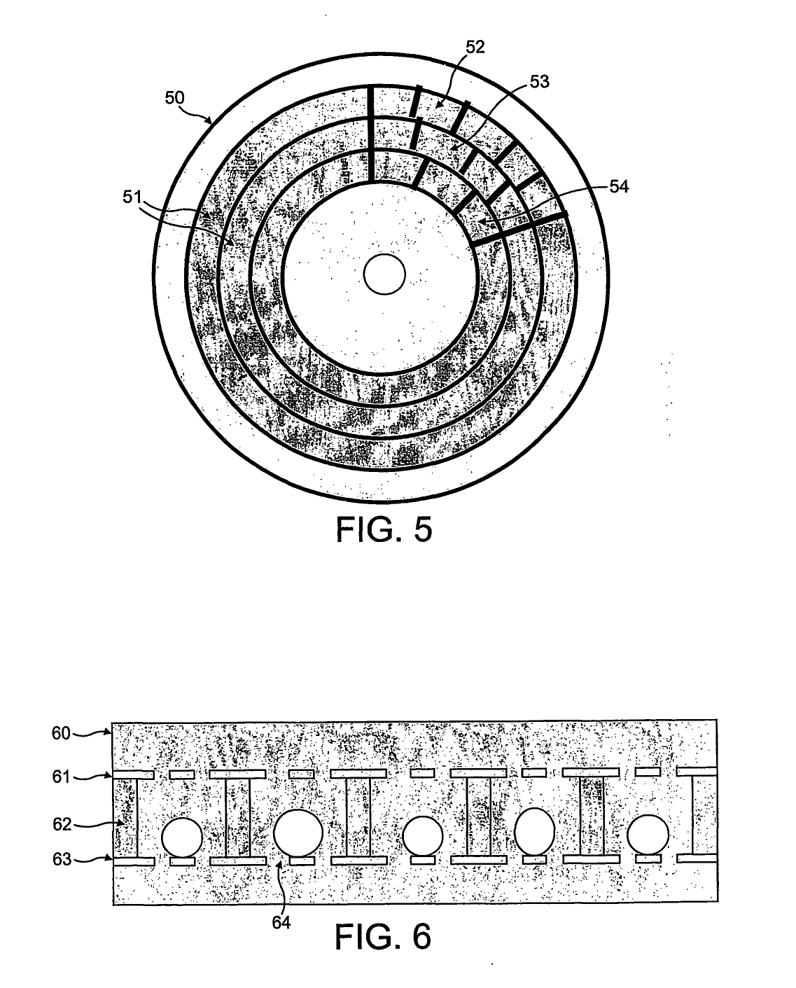

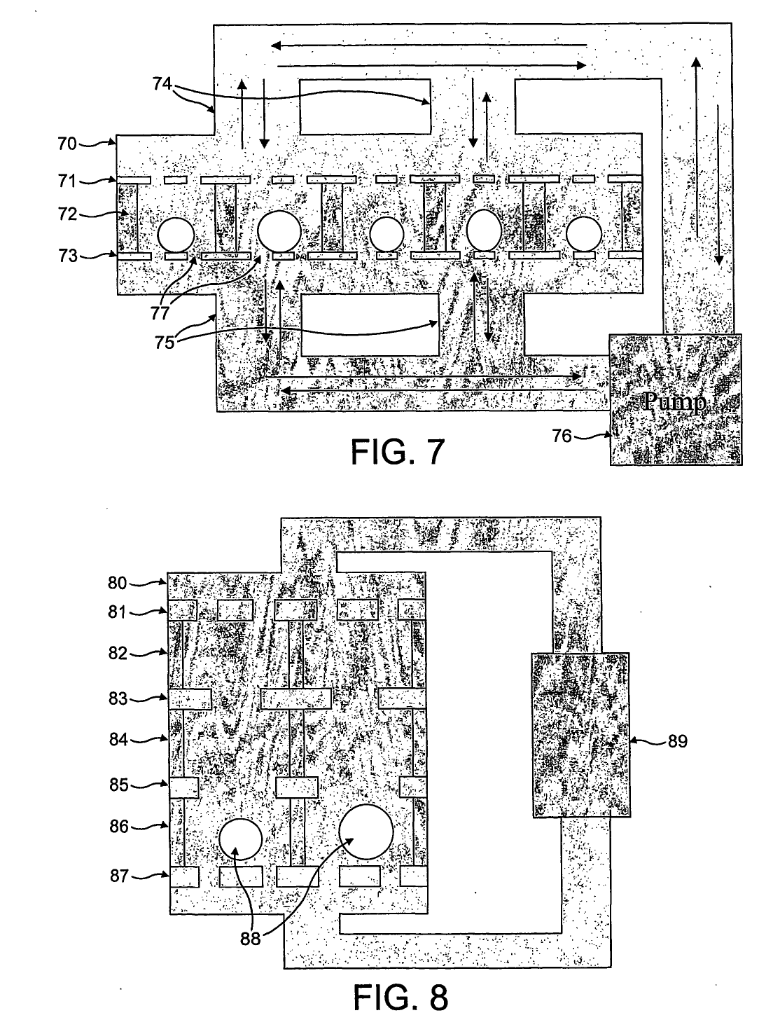

The micro chambers can be arranged in a DISK format similar to that found in musical compact disks (FIG. 5.) The chambers (52,53,54) are designed as described herein but arranged in concentric circles (51) to form a disc (50). The combination of micro chamber and DISK technology can be used to incorporate centrifugation and precise location of all individual chambers for quality assessment of the follicles, oocytes or embryos. Individual disks can be taken out of growth/maturation/culture devices and accompanying conditions (FIGS. 6, 7) at regular intervals.

A cell/follicle/oocyte/embryo growth/maturation device can be constructed in different ways depending on the objectives. Growth/maturation or certain phases of the process can be done in a static device as depicted in FIG. 6. The top of the arrangement (60) may also contain a sieve element (61) that will cover the microchambers (62). The micro chamber arrangement (60) may also include a second sieve element (63). Sieve elements 61 and 63 have holes (64). Without a second sieve element (63) the space below the microchambers (62) needs to be narrow enough to prevent the follicles, embryos or oocytes from leaving the microchambers. The arrangement (60) can be made to vibrate, slowly rotate or turn 180 degrees and back again in order to avoid the oocytes/embryos sticking to the wall or bottom.

In a further embodiment of FIG. 6 a combined microchamber and 3D sieve arrangement could be formed by assembling successive layers of spacers and drilled (sieve) elements sized to allow flow of media or small cells between layers but not to allow escape of larger cells or embryos.

In a further embodiment the microchambers (62) may contain holes within the connecting walls. This allows medium to flow in and out of each individual microchamber from the sides and/or the top and bottom.

FIG. 7 shows a microchamber arrangement (70) that allows refreshing or changing of the culture medium. The media can be refreshed and/or changed from either direction, depending on the flow rate. A filter in the pump (76) can be designed to remove toxic molecules while the favourable molecules remain in the system. Inlets (74) and outlets (75) are evenly spread over top and bottom to give a uniform flow rate across the total device. Reversing the flow of the medium can prevent the oocyte/embryo from sticking to the wall or bottom. The microchamber arrangement (70) may incorporate sieve elements (71,73), which have holes (77), and form the top and bottom of the individual microchamber (72). In some arrangements the top sieve (71) or bottom sieve (73) can be omitted.

Quality control can be based on size using apparatus as shown in FIG. 8. Here the arrangement consists of three layers of microchambers (82, 84,86) arranged on top of each other. The bottom layer (86) holds the maturing follicles, oocytes or embryos (88). The other layers (82, 84) are put on top of the base layer (86). The sieve elements (81,83,85,87) form the top and bottom of the microchamber arrangement (80) and separate the layers of the microchambers (82,84,86). The middle layer (84) is a type II arrangement and has the centre hole of a size so that only smaller oocytes can pass through the separation device. This results in the sorting of the oocytes by size. The number of layers of microchambers can be increased and by using different sizes of the holes in the centre, the oocytes can be sorted into more than two classes. Further growth and maturation of the oocytes (FIG. 7) can then be class (size, stage of development, quality) specific. The pump (89) can be used to add several substances such as hormones, growth factors, peptides etc. to the medium at the appropriate stage of development.

In Vitro Fertilization

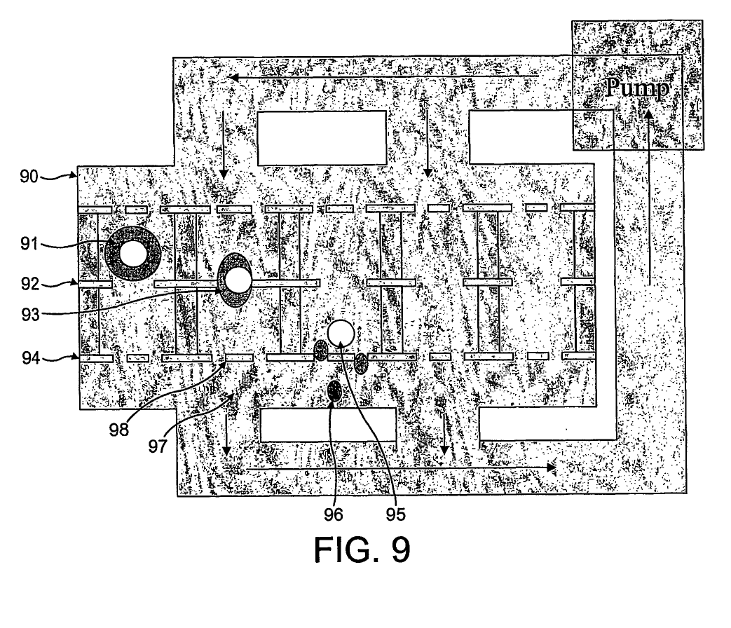

The cumulus cells need to be removed from the COC before fertilisation can take place. This process can be carried out by a microchamber arrangement as depicted in FIG. 9. Here the arrangement consists of two layers of microchambers on top of each other. The bottom sieve element (92) of the first layer has one larger hole (type II arrangement) while the top sieve element (98) and the bottom sieve element (94) of the second layer has several smaller holes (type I arrangement). The matured oocytes and the associated cumulus cells (91, 93) are squeezed through the larger hole of the type II arrangement (92) removing the surrounding cells. The cumulus cells (96) are then sucked away through the smaller holes of the type I arrangement (94) to leave the oocyte (95). The flow can be reversed to repeat the process if necessary. The top and bottom sieves (98, 94) can be removed, in which case the space above (90) and below (97) the microchamber arrangement is narrow enough to prevent the oocytes leaving the microchambers. In this configuration (sieves (98, 94) removed) the cumulus cells could be removed by high speed flushing of media through the microchamber by arranging that the space above (90) or below (97) the chamber is large enough to permit small cells and debris to pass yet small enough to prevent the escape of large cells or embryos.

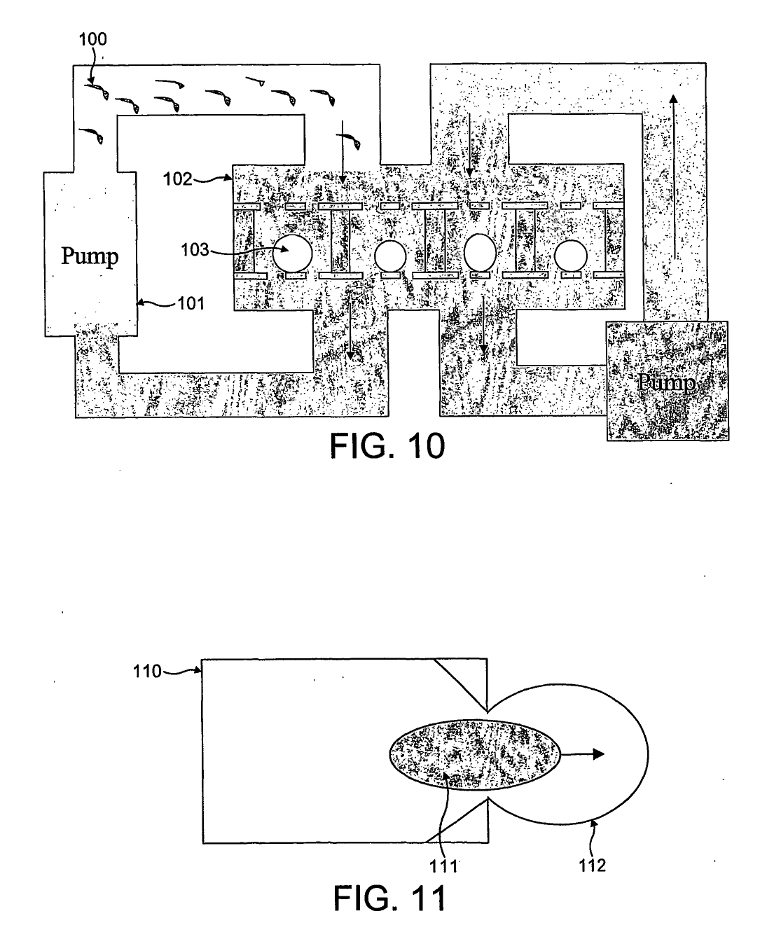

FIG. 10 depicts arrangements for carrying out in vitro fertilisation. Individual sperm cells (100) can be added to the chambers (by robot and/or laser technology) so that in vitro fertilisation can take place. This process can be repeated several times at pre-determined intervals. Procedures can be optimised in order to maximise the fertilisation rate and to minimise polyspermy. It is also possible to add semen to the medium before it flows into the device that holds the mature oocytes (103), or by having a double inlet system as depicted in FIG. 10. Semen is diluted to a concentration so that only a small number of sperm cells enter any one micro chamber. Semen is put into the second pump system (101). The semen (100) and diluent flow into the micro chamber arrangement (102) until the contents of the arrangement (102) are replaced once. After a certain interval the process is repeated. The length of the interval, the number of rounds of semen input and the concentration of sperm cells can be varied to produce optimal conditions. The semen can be a mix of both X and Y types, or single sex progeny can be produced by using one sex-selected type.

FIG. 11 illustrates a different microchamber (110) design. It has an additional section (112) that has the dimension equal to the size of a matured oocyte. Oocytes that have been enucleated (DNA removed or made dysfunctional), or normal oocytes (111), are pushed into the compartment (112) by centrifugation. Donor cells, sperm cells or other material can be injected into the enucleated or normal oocytes by a micro injection robot. Similar procedures can be used for genetic modification by injecting foreign DNA into pronuclear stage embryos or for taking biopsies for DNA and other analysis.

Embryo Culture

The methods for embryo culture, with or without flow, sorting on size and rotating the device are similar to the procedures described for oocyte maturation. Video image analysis can be used to monitor the development of the embryos. The time pattern of moving from the one-cell stage to the 2-cell stage, 4-cell stage etc. can be monitored. This time pattern in combination with other parameters can be used to develop embryo quality indexes. A microscope connected to a video camera can scan the microchamber arrangement at regular intervals. Software can be developed to define and measure the embryos. Sequential images can be used to monitor activity (e.g. by change of shape) and to measure development (e.g. size and colour). The concentration of metabolite build-up and/or nutrient uptake in the individual chamber can be used to measure the metabolism of individual embryos in a non-flow situation. This monitoring could involve taking micro samples (e.g. using robots) for analysis, tracking colour reactions of the medium which would be based on concentrations of metabolites in the chambers or any other method. The linked computer is used to analyse all the data and to predict per chamber the developmental competence of the embryos. The quality of embryo development in the total device can be evaluated by taking samples of the medium at the outlet of the device.

In an alternative embodiment to FIG. 10 the bottom and or top of the microchambers could be selectively closed or opened to prevent or allow flow of media and growth products between microchambers.

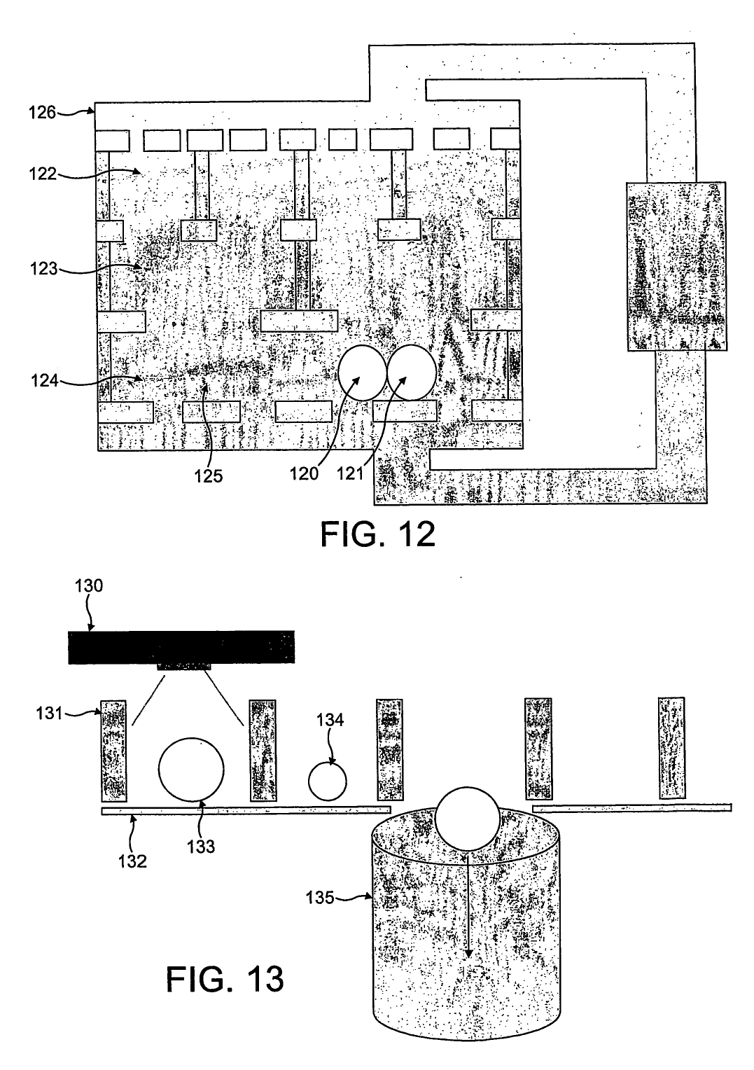

FIG. 12 illustrates how the technology can be used for embryo splitting. In this arrangement (126) there are three microchamber layers (122, 123, 124), with the bottom layer (124) consisting of relatively large microchambers (125) in which the embryo (120/121) resides. Enzymes that remove the zona pollucida are added to the medium that flows through the chamber (125) to separate the blastomeres from a 2 cell stage embryo (or 4 or 8 cell stage). The individual blastomeres (120/121) are separated into different chambers by inversion of the device, after which further development from the one cell to the 2 cell stage etc. can take place. This process can be repeated several times. This will result in a number of identical copies of the single original embryo. One copy can be used for sex determination in case the semen was not sex selected (X or Y sperm cells only). At some stage of development an artificial zona pellucida can be created through encapsulation.

In addition to the selection based on size other quality assessments can be performed based on video image and other types of analyses, assisting in the selection of appropriate follicles, oocytes and/or embryos. (FIG. 13) A device (130), eg. a video camera linked to microscope or other type of image capturing system such as ultra sound, can be used to evaluate the quality of developing follicles, oocytes, fertilised oocytes, cultured embryos or encapsulated embryos. Once the quality has been assessed, selected follicles, or oocytes/embryos (133, 134) can be flushed into a device below (135), from microchambers (131) via an adjustable sieve element (132). This device (135) can be a straw (for embryo shipment or transfer to recipient females) or another microchamber arrangement for further development of the follicle, oocyte/embryo.

Encapsulation

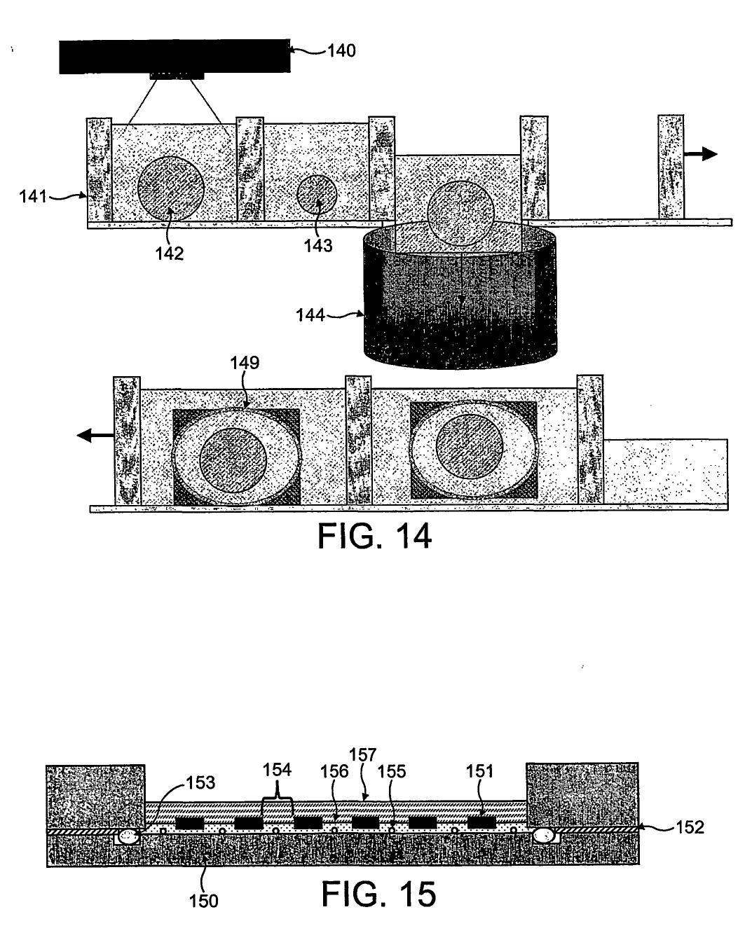

High quality embryos can be selected from the micro-chambers by a robot and transferred into an encapsulation system or an arrangement can be used as illustrated in FIG. 14.

Encapsulated embryos are transported in a temperature controlled device. It is also possible to do the encapsulation after transport. Embryos can be encapsulated with the biodegradable materials alginate-calcium chloride, agar, gelatine, or some other suitable substance. The higher the concentration of encapsulation material used, the more impermeable the matrix becomes, which results in a lengthened period of time before the microcapsule is compromised. Both zona-free and “normal” embryos can be microencapsulated. Encapsulation of zona-free embryos is important for cloning based on embryo splitting or for frozen/thawed zone-free embryos. Capsules can be made to be of almost any size and have ranged from about 20 μm to greater than 1 mm in diameter.

The encapsulation system (FIG. 14) involves dispensing (140) a small volume of 3% sodium alginate into higher (taller) micro-chambers (141). The embryo (142,143) surrounded by culture medium is then added. This is followed by a second small volume of sodium alginate. The sodium alginate/embryo mixture is then held above a solution of CaCl₂ (144) and expelled. The resulting microcapsule (149) should be approximately 1 mm in diameter and is ready for transport or transfer to a recipient animal. This technique is based on the procedure described by Adaniya et al. 1993. The present invention provides continuous control over the follicular growth, maturation/fertilisation/manipulation and culture environment of follicles, oocytes and/or embryos. It also allows for quality control enabling further processing based on size and/or other quality parameters. The process can be automated and thus standardised, which will increase production and efficiency.

The cross-sectional view of a micro-chamber arrangement in FIG. 15 is comprised of the base plate (150), well plate (151), spacers (152), and a sealing O-ring (153). Individual micro-chambers (154) containing the cultured object (155) in medium (156) are shown. An optional mineral oil overlay (157) covers the culture medium.

### EXAMPLES

### Example 1

Oocyte maturation (metaphase II (MII)) and cumulus expansion was compared after culture for 44-46 h in 5 μl micro-drops under oil and in 25 well MCT device (FIG. 15) with a shared volume (245 μl/MCT).

| | | | Cat. ≦ 2 | Cat. 3 |
|---|---|---|---|---|
| Type of | Culture | M II | cumulus | cumulus |
| culture | volume | (%) | expansion | expansion |
| Culture drops | 5 μl/oocyte | 78 | 100% | — |
| Under oil | | | | |
| MCT with | 245 μl/MCT | 77 | 100% | — |
| shared volume | (5 μl/oocyte) | | | |

Culture of oocytes in 25 well MCT device with a shared volume, a maturation rate of 77% was obtained. The MCT oocyte maturation rate was comparable to those oocytes (78%) cultured individually in 5 μl micro-drops under oil. All of the oocytes cultured individually in MCT and micro-drops showed cumulus expansion category of ≦2 in comparison to 26% in category ≦2 and 74% in category 3 for group culture.

### Example 2

Oocyte maturation and cumulus expansion was examined using micro-drops and MCT device (FIG. 15) with a shared volume.

| | | | Cat. ≦ 2 | Cat. 3 |
|---|---|---|---|---|
| Type of | | M II | cumulus | cumulus |
| culture | Culture volume | (%) | expansion | expansion |
| Culture drops | 5 μl/oocyte | 73 | 100% | — |
| Under oil | | | | |
| MCT with | 245 μl/MCT | 76 | 100% | — |
| shared volume | (5 μl/oocyte) | | | |

The maturation rate in MCT was 76% while the 5 μl micro-drop control was 73%. Cumulus expansion in both groups ranged up to category 2 and was similar.

### Example 3

Similar to Example 1, oocyte maturation and cumulus expansion was compared after culture for 44-46 h in 5 μmicro-drops under oil and in 25 well MCT device FIG. 15) with a shared volume (245 μl/MCT).

| | | | Cat. ≦ 2 | Cat. 3 |
|---|---|---|---|---|
| Type of | | M II | cumulus | cumulus |
| culture | Culture volume | (%) | expansion | expansion |
| Culture drops | 5 μl/oocyte | 85 | 100% | — |
| Under oil | | | | |
| MCT with | 245 μl/MCT | 71 | 100% | — |
| shared volume | (5 μl/oocyte) | | | |

By using the same 25 well MCT prototype with 5 μl volume/well, a maturation rate of 71% was obtained while 85% of oocytes reached M II stage in 5 μl micro-drops under oil. None of the oocytes cultured in MCT and micro-drops showed cumulus expansion beyond category 2 level.

### Example 4

Two 49 small well MCT devices (FIG. 15) with shared volume were used in this experiment. The volume per MCT well was 5 μl (total volume 245 μl/MCT device). One device was used to culture oocytes aspirated from 3-6 mm follicles while the other was used for oocytes collected from >6 mm follicles was 67% at category ≦2 and 33% at category 3.

| | | | Cat. ≦ 2 | Cat. 3 |
|---|---|---|---|---|
| | | | cumulus | cumulus |
| Oocytes source | Culture volume | M II (%) | expansion | expansion |
| 3-6 mm follicles | 5 μl/oocyte | 84 | 98% | 2% |
| >6 mm follicles | 5 μl/oocyte | 85 | 67% | 33% |

Following culture the percentage of oocytes reaching MII was 84 and 85% for those obtained from 3-6 and >6 mm follicles, respectively. Cumulus expansion for the oocytes from 3-6 mm follicles was 98% (category ≦2) and 2% (category 3), while expansion in the oocytes from >6 mm follicles was much better (67% at category ≦2 and 33% at category 3). Data suggests that MCT device itself does not appear to inhibit cumulus expansion or maturation.

### Example 5

Using 3 different types of MCT devices (FIG. 15), oocytes were matured individually in 7 μl volumes for 44-46 h. The use of the term individual volume in examples 5-8 refers to an MCT device (FIG. 15) that does not have the interconnections between individual micro-chambers. The terms small and large wells in the same examples refers to the diameter of each individual micro-chamber well (164).

| | | | Cat. ≦ 2 | |
|---|---|---|---|---|
| Type of MCT | | M II | cumulus | Cat. 3 cumulus |
| device | Culture volume | (%) | expansion | expansion |
| Small well (49 | 343 μl/MCT | 87 | 63% | 37% |
| wells), Shared | (7 μl/oocyte) | | | |
| volume | | | | |
| Small well, | 7 μl/oocyte | 83 | 36% | 64% |
| individual volume | | | | |
| Large well (49 | 343 μl/MCT | 85 | 67% | 33% |
| wells), Shared | (7 μl/oocytes) | | | |
| volume | | | | |

For a 7 μl well volume, maturation rates (MII) ranged from 83-87% for three types MCT devices. In contrast to previous studies, 33-64% of oocytes exhibited category 3 cumulus expansion. It should be noted in this study that foetal calf serum FCS in IVM medium was replaced by follicular fluid and may have assisted in expansion of cumulus cells.

### Example 6

Using different types of MCT devices (FIG. 15), oocytes were matured individually either in 7 or 10 μl volumes for 44-46 h.

| | | | Cat. ≦ 2 | Cat. 3 |
|---|---|---|---|---|
| Type of MCT | Culture | M II | cumulus | cumulus |
| device | volume | (%) | expansion | expansion |
| Small well, | 7 ul/oocyte | 91 | 69% | 31% |
| Shared volume | | | | |
| Small well, | 7 ul/oocyte | 95 | 59% | 41% |
| individual volume | | | | |
| Large well, | 7 ul/oocyte | 95 | 52% | 48% |
| individual volume | | | | |
| Large well, | 10 ul/oocytes | 100 | 71% | 29% |
| individual volume | | | | |
| Small well, | 10 ul/oocytes | 96 | 69% | 31% |
| individual | | | | |
| volume | | | | |

For oocytes matured in 7 μl well volumes, maturation rates of 91-95% were obtained. These same groups, 31-48% of oocytes exhibited category 3 cumulus expansion. For oocytes cultured in small and large well individual volumes (10 μl per well), 96-100% completed maturation to M II stage with similar proportion (29-31%) of oocytes showing category 3 cumulus expansion.

### Example 7

Same procedure as example 6.

| | | | Cat. ≦ 2 | Cat. 3 |
|---|---|---|---|---|
| Type of MCT | Culture | M II | cumulus | cumulus |
| device | volume | (%) | expansion | expansion |
| Small well, | 7 ul/oocyte | 82 | 61% | 39% |
| Shared volume | | | | |
| Small well, | 7 ul/oocyte | 91 | 57% | 43% |
| individual volume | | | | |
| Large well, | 7 ul/oocyte | 94 | 57% | 43% |
| individual volume | | | | |
| Large well, | 10 ul/oocytes | 95 | 52% | 48% |
| individual volume | | | | |
| Small well, | 10 ul/oocytes | 91 | 39% | 61% |
| individual volume | | | | |

Results are very similar to Example 6. For oocytes matured in 7 μL well volumes, maturation rates of 82-94% were obtained. These same groups, 39-43% of oocytes exhibited category 3 cumulus expansion. For oocytes cultured in small and large well MCT protypes individual in 10 μl volumes, 91-95% completed maturation to M II stage with similar proportion (48-61%) of oocytes showing category 3 cumulus expansion.

### Example 8

Oocytes were cultured individually in MCT (FIG. 15) with individual volume (10 μl/well) or shared volume (490 μl/49 well MCT device) for 44-46 h.

| | | | Cat. ≦ 2 | Cat. 3 |
|---|---|---|---|---|
| Type of MCT | Culture | M II | cumulus | cumulus |
| device | volume | (%) | expansion | expansion |
| Large well MCT with | 7 μl/oocyte | 89 | 76% | 24% |
| Individual volume | | | | |
| (10 μl/well) | | | | |
| Large well MCT with | 7 μl/oocyte | 80 | 69% | 31% |
| shared volume (490 μl/49 | | | | |
| well MCT) | | | | |

Culture of oocytes in MCT protypes in individual volumes or with shared volumes did not affect the M II rate or cumulus expansion of category 3.

### Example 9

This experiment was the first attempt at using the large well, shared volume MCT devices (FIG. 15) for performing IVF by using frozen-thawed semen. The volume of medium used for IVF was 10 μl per oocyte and the concentration of sperm was 0.75×10⁵/ml. An oil overlay was used to cover the IVF medium in MCT devices. Sperm-oocyte were co-incubated for 10 h. A penetration rate of 84% was achieved. Polyspermy rates were relatively high at 63%. The control groups (100 μl drops under oil) gave penetration rates of 78% while polyspermy was 45% after 5 h co-incubation.

### Example 10

This experiment was the second attempt at using MCT (FIG. 15) for IVF. This attempt was made without using a mineral oil overlay. Instead, a mini-humidified environment was created by placing the MCT devices into 100×15 mm petri dishes containing water. The volume of IVF medium used was 10 μl and the sperm concentration was 0.5×10⁵/ml. Sperm-oocyte were co-incubated for 5 h only. The penetration rate and polyspermy obtained after IVF in MCT device was 55% and 19%, respectively. Control IVF (100 μl drops under oil) yielded a penetration rate of 73% and 41% polyspermy.

### Example 11

This preliminary study was carried out to examine the ability of IVM-IVF derived pig embryos to develop individually in small culture volumes. At 48 h after IVF, 2-4 cell embryos were selected and cultured for 96 h in 10 μl drops under oil, or in the MCT device (FIG. 15) with individual volumes of 10 μl/well. Some of the embryos cultured in drops and all of the embryos cultured in the individual MCT device were transferred to a fresh culture medium at 48 h of the 96 h culture.

| | Blastocyst at 96 | Blastocyst at 96 |
|---|---|---|
| | h of continuous | h with medium |
| Type of culture system | culture | change at 48 h |
| 10 μl drops covered | 58% | 60% |
| with oil (control) | | |
| Large well MCT | No data | 43% |
| device with individual | | |
| volume (10 μl) | | |

No difference in embryo development was observed in drop culture with or without medium change (58 vs 60%). In the MCT device with individual wells, 43% of embryos developed to blastocyst stage following medium change at 48 h. The results indicate that successful embryo culture is possible in MCT devices.

Summary of MCT Data on IVM/IVF and EC

IVM

Preliminary in vitro maturation (IVM) studies carried out for 44-48 h using MCT prototypes (FIG. 15) having small-wells or large-wells with individual or shared volume (7-10 ul/well/oocyte) resulted in 85-100% of oocytes completing nuclear maturation to metaphase II (M II) stage. At the end of IVM, 31-48% of oocytes showed category 3 cumulus expansion as described by Abeydeera et al. (2000). The basic oocyte maturation medium is either tissue culture medium (TCM) 199 or North Carolina State University (NCSU) 23 Medium and was supplemented with FCS or follicular fluid, growth factors, a thiol compound and gonadotropins. Results demonstrate that MCT can be used for IVM. Optimisation incorporating flow is required to further improve the results.

IVF

MCT prototypes (FIG. 15) with large-wells with shared volume was used for in vitro fertilisation of IVM oocytes placed individually in 10 ul volumes. IVF medium was essentially the same as that described previously by Abeydeera et al. (2000). After 10 h sperm-oocyte co-incubation, 84% of oocytes were penetrated with 63% polyspermy. In another study, penetration rate and polyspermy was 55% and 19%, respectively, following 5 h of sperm-oocyte co-incubation. Results demonstrate that MCT can be used for IVF. System can be used to optimise the UVF procedure in order to improve penetration rate and reduce polyspermy.

EC

Culture of IVM-IVF derived 2-4 cell embryos individually in 10 ul volumes in large-well MCT prototype (FIG. 15) resulted in 43% of blastocyst development after 96 h culture with a transfer to fresh medium at 48 h. These results show that embryo culture is possible when using MCT.

### Reference List

Abeydeera L R, et al. Development and viability of pig oocytes matured in a protein-free medium containing epidermal growth factor. Theriogenology 2000; 54:787-797.

Adaniya G K et al. First pregnancies and live births from transfer of sodium alginate encapsulated embryos in a rodent model. Fert Steril 1993; 59(3):652-656.

Beebe L F S, et al. Piglets born from vitrified zona-intact blastocysts. Theriogenology 2000; 53:249.

Figueiredo et al., State of the art of manipulation of oocytes enclosed in preantral follicles. Embryo Transfer Newsletter 2000; 18:11-15.

Hirao et al. In vitro growth and maturation of pig oocytes. J. Reprod & Fert 1994; 100:333-339.

McCaffery F H et al. Culture of bovine prenatral follicles in a serum-free system: Markers for assessment of growth and development. Biol Reprod 2000; 63:267-273;

Raty S, et al. Culture in microchannels enhances in vitro embryonic development of preimplantation mouse embryos. Theriogenology 2001; 55:241.

Telfer E E et al. In vitro development of oocytes from porcine and bovine primary follicles. Mol Cell Endo 2001;163:117-123

Walters E M et al. In vitro maturation of pig oocytes in polydimethylsiloxane (PDMS) and silicon microfluidic devices. Theriogenology 2001; 55(1): 49

Wu et al. In vitro growth, maturation, fertilisation, and embryonic development of oocytes from porcine preantral follicles. Biology of Reproduction 2001; 64:375-381.

1. Apparatus for handling and/or treatment of cells, in particular follicles, oocytes and/or embryos comprising of at least one
microchamber arrangement containing a plurality of microchambers, each optionally comprising one or more sieve elements, wherein the micro chamber arrangement further incorporates a pump that controls the recirculation of medium and optionally devices to measure and/or regulate characteristics of the medium, and/or introduce spermatozoa.

2. Apparatus as claimed in claim 1 further comprising a chamber containing a plurality of sieve elements arranged in succession within the chamber, wherein each successive sieve element has pores of a smaller dimension than those in the preceding sieve element, connected to a pump to maintain a circulatory flow of medium, wherein said sieve elements separate primordial follicles, preantral follicles or Cumulus-Oocyte-Complexes (COCs) from ovarian debris and sorts the follicles according to size.

3. Apparatus as claimed in claim 1 or claim 2 wherein the characteristics are any one or more of pH, osmolarity, carbon dioxide levels, and temperature.

4. Apparatus as claimed in claim 1 wherein the microchamber sieve elements are adjustable, such that the pore size can be altered, or the pores can be closed.

5. Apparatus as claimed in claim 1 wherein the microchamber sieve elements have pores of different dimension.

6. Apparatus as claimed in claim 1 wherein the microchamber arrangement comprises a series of layers of microchambers.

7. Apparatus as claimed in claim 6 wherein sieve elements associated with each layer of microchambers form a plurality of sieve elements arranged in succession, wherein each successive sieve element has pores of a larger pore dimension than those in the preceding sieve element.

8. Apparatus as claimed in claim 6 wherein sieve elements associated with each layer of microchambers form a plurality of sieve elements arranged in succession, wherein each successive sieve element has pores of a smaller pore dimension than those in the preceding sieve element.

9. Apparatus as claimed in claim 6 successive layers contain microchambers of decreasing dimension.

10. Apparatus as claimed in claim 1, wherein the connecting walls of said microchambers contain holes to allow the flow of medium in and/or out of each individual microchamber from the sides.

11. Apparatus as claimed in claims 1 wherein the microchamber arrangement is linked to an imaging means linked to an image capturing device.

12. Apparatus as claimed in claim 10 wherein the imaging means is a video camera and the image capturing device is a microscope or uses ultra sound to generate a quality index based on visual assessment and other parameters such as medium pH, medium osmolarity, medium temperature, and/or oocyte/embryo metabolism.

13. Apparatus as claimed in claim 11 wherein the imaging is linked to other devices that monitor characteristics such as medium pH, temperature, osmolarity and/or oocyte/embryo metabolism to generate a quality index for each said oocyte/embryo.

14. Apparatus as claimed in claim 4 wherein the microchamber arrangement is positioned above a transfer device.

15. Apparatus as claimed in claim 13 wherein the transfer device is a second microchamber arrangement or straw.

16. Apparatus as claimed in claim 1 wherein the micro chamber arrangement further incorporates a second pump system.

17. Apparatus as claimed in claim 15 wherein the second pump regulates the introduction of a small number of spermatozoa into the micro chambers.

18. Apparatus as claimed in claim 6 wherein the microchamber arrangement further incorporates oocyte/embryo encapsulation means.

19. Apparatus as claimed in claim 1 wherein each microchamber consists of two sub-chambers.

20. Apparatus as claimed in claim 18 wherein the subchamber is formed to have dimensions equivalent to or greater than a mature oocyte.

21. The use of the apparatus as claimed in claim 1 in one or more of:
(a) Separation of follicles from ovaries
(b) Growth of follicles
(c) Removal of COCs from follicles
(d) Maturation of COCs (IVM)
(e) Removal of cumulus cells from COC's
(f) Oocyte enucleation
(g) In vitro fertilisation
(h) Nuclear transfer (NT)/cloning
(i) Embryo culture
(j) Splitting embryos
(k) Sorting embryos and or oocytes according to size
(l) Encapsulation
(m) Transport of embryos from the production facility to the site of embryo transplantation
(n) Cell culture.

22. The use as claimed in claim 20 when the oocytes or embryos or sperm are from mammals or aquatic species.
